SEQUENCE LISTING (15 sequences)

SEQ ID NO: 1 - DNA, 723 bp, Artificial sequence
  Isolation and characterization of plasmid DNA encoding a scFV human antibody fragment, called SC02-40 7, from synthetic CDR3 regions combined with a collection of pHEN1 phagemid-derivon bacteriophage
  CDS (1)..(723): Coding sequence for phage antibody SC02-407
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaaggacacgctgtatctgcaaatgagcagcctgaaaaccgaggacacggccgtgtattactgtgcaagagggtcgttgttgcagttttggggccaaggtaccctggtcaccgcctcgagaggtggaggcggttcaggcggaggtggctctggcggtggcggatcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggtgcggccgca

SEQ ID NO: 2 - protein, 241 aa, Artificial sequence
  Amino acid sequence of phage antibody SC02-407
EVQLVESGGGLVQPGGSLRLSCAASGFTFSSYAMSWVRQAPGKGLEWVSAISGSGGSTYYADSVKGRFTISRDNSKDTLYLQMSSLKTEDTAVYYCARGSLLQFWGQGTLVTASRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAA

SEQ ID NO: 3 - protein, 6 aa, Artificial sequence
  heavy chain CDR3 variable region of  phage antibody SC02-407
GSLLQF

SEQ ID NO: 4 - DNA, 6778 bp, Artificial sequence
  IgG1 expression vector pSyn-C03-HCgamma1
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagctggcctggatggcctgactctcttaggtagccttgcagaagttggtcgtgaggcactgggcaggtaagtatcaaggttacaagacaggtttaaggagatcaatagaaactgggcttgtcgagacagagaagactcttgcgtttctgataggcacctattggtcttactgacatccactttgcctttctctccacaggtgtccactcccagttcaattacagctcgccaccatggcctgccccggcttcctgtgggccctggtgatcagcacctgcctggaattcagcatgagcagcgctagcaccaagggccccagcgtgttccccctggcccccagcagcaagagcaccagcggcggcacagccgccctgggctgcctggtgaaggactacttccccgagcccgtgaccgtgagctggaacagcggcgccttgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaaacgcgtggagcccaagagctgcgacaagacccacacctgccccccctgccctgcccccgagctgctgggcggaccctccgtgttcctgttcccccccaagcccaaggacaccctcatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctcaccgtgctgcaccaggactggctgaacggcaaggagtacaagtgcaaggtgagcaacaaggccctgcctgcccccatcgagaagaccatcagcaaggccaagggccagccccgggagccccaggtgtacaccctgccccccagccgggaggagatgaccaagaaccaggtgtccctcacctgtctggtgaagggcttctaccccagcgacatcgccgtggagtgggagagcaacggccagcccgagaacaactacaagaccaccccccctgtgctggacagcgacggcagcttcttcctgtacagcaagctcaccgtggacaagagccggtggcagcagggcaacgtgttcagctgcagcgtgatgcacgaggccctgcacaaccactacacccagaagagcctgagcctgagccccggcaagtgataatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggatcgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 6616 bp, Artificial sequence
  Expression Construct, called pSyn-C04-Vlambda3, used to obtain IgG1 antibody expression
cgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgttaattaacatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaatcgatgactctcttaggtagccttgcagaagttggtcgtgaggcactgggcaggtaagtatcaaggttacaagacaggtttaaggagatcaatagaaactgggcttgtcgagacagagaagactcttgcgtttctgataggcacctattggtcttactgacatccactttgcctttctctccacaggtgtccactcccagttcaattacagctcgccaccatggcctgccccggcttcctgtgggccctggtgatcagcacctgcctcgagttttccatggcttcctccgagctgacccaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggtgagtcgcggccgcaagcttaccgtgctgggccagcccaaggccgctcccagcgtgaccctgttccccccctcctccgaggagctgcaggccaacaaggccaccctggtgtgcctcatcagcgacttctaccctggcgccgtgaccgtggcctggaaggccgacagcagccccgtgaaggccggcgtggagaccaccacccccagcaagcagagcaacaacaagtacgccgccagcagctacctgagcctcacccccgagcagtggaagagccaccggagctacagctgccaggtgacccacgagggcagcaccgtggagaagaccgtggcccccaccgagtgcagctaatagacttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggccatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 6 - DNA, 52 bp, Artificial sequence
  Oligonucleotide 5L-A
acctgtctcgagttttccatggcttcctccgagctgactcaggaccctgctg

SEQ ID NO: 7 - DNA, 44 bp, Artificial sequence
  Oligonucleotide 3L-B
gaccaagctgaccgtcctaggtgagtcgcggccgctaaggaaaa

SEQ ID NO: 8 - DNA, 50 bp, Artificial sequence
  Oligonucleotide 5H-B
acctgtcttgaattctccatggcccaggtgcagctgcaggagtccggccc

SEQ ID NO: 9 - DNA, 47 bp, Artificial sequence
  Oligonucleotide Sy3H-a reversed
ggggccagggcaccctggtgaccgtctccagcgctagcaccaagggc

SEQ ID NO: 10 - DNA, 1398 bp, Artificial sequence
  The nucleotide sequence of the heavy chain of the IgG1 antibody, called CR2407, derived from pSyn-CO4-Vlambda3 expression vector
  CDS (1)..(1398): Coding sequence for the heavy chain of IgG1 antibody CR2407
  sig_peptide (1)..(63): Signal peptide for the heavy chain of IgG1 antibody CR2407
atggcctgccccggcttcctgtgggccctggtgatcagcacctgcctggaattctccatggccgaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaaggacacgctgtatctgcaaatgagcagcctgaaaaccgaggacacggccgtgtattactgtgcaagagggtcgttgttgcagttttggggccagggcaccctggtgaccgtctccagcgctagcaccaagggccccagcgtgttccccctggcccccagcagcaagagcaccagcggcggcacagccgccctgggctgcctggtgaaggactacttccccgagcccgtgaccgtgagctggaacagcggcgccttgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaaacgcgtggagcccaagagctgcgacaagacccacacctgccccccctgccctgcccccgagctgctgggcggaccctccgtgttcctgttcccccccaagcccaaggacaccctcatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctcaccgtgctgcaccaggactggctgaacggcaaggagtacaagtgcaaggtgagcaacaaggccctgcctgcccccatcgagaagaccatcagcaaggccaagggccagccccgggagccccaggtgtacaccctgccccccagccgggaggagatgaccaagaaccaggtgtccctcacctgtctggtgaagggcttctaccccagcgacatcgccgtggagtgggagagcaacggccagcccgagaacaactacaagaccaccccccctgtgctggacagcgacggcagcttcttcctgtacagcaagctcaccgtggacaagagccggtggcagcagggcaacgtgttcagctgcagcgtgatgcacgaggccctgcacaaccactacacccagaagagcctgagcctgagccccggcaag

SEQ ID NO: 11 - protein, 466 aa, Artificial sequence
  The amino acid sequence of the heavy chain of the IgG1 antibody CR2407
MACPGFLWALVISTCLEFSMAEVQLVESGGGLVQPGGSLRLSCAASGFTFSSYAMSWVRQAPGKGLEWVSAISGSGGSTYYADSVKGRFTISRDNSKDTLYLQMSSLKTEDTAVYYCARGSLLQFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 759 bp, Artificial sequence
  The nucleotide sequence of the light chain of the IgG1 antibody CR2407
  sig_peptide (1)..(63): Signal peptide for the light chain of IgG1 antibody CR2407
  CDS (1)..(759): Coding sequence for the light chain of IgG1 antibody CR2407
atggcctgccccggcttcctgtgggccctggtgatcagcacctgcctcgagttttccatggcttcctccgagctgacccaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggtgagtcgcggccgcaagcttaccgtgctgggccagcccaaggccgctcccagcgtgaccctgttccccccctcctccgaggagctgcaggccaacaaggccaccctggtgtgcctcatcagcgacttctaccctggcgccgtgaccgtggcctggaaggccgacagcagccccgtgaaggccggcgtggagaccaccacccccagcaagcagagcaacaacaagtacgccgccagcagctacctgagcctcacccccgagcagtggaagagccaccggagctacagctgccaggtgacccacgagggcagcaccgtggagaagaccgtggcccccaccgagtgcagctaatagacttaagtttaaaccgc

SEQ ID NO: 13 - protein, 253 aa, Artificial sequence
  The amino acid sequence of the light chain of the IgG1 antibody CR2407
MACPGFLWALVISTCLEFSMASSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGESRPQAYRAGPAQGRSQRDPVPPLLRGAAGQQGHPGVPHQRLLPWRRDRGLEGRQQPREGRRGDHHPQQAEQQQVRRQQLPEPHPRAVEEPPELQLPGDPRGQHRGEDRGPHRVQLIDLSLNR

SEQ ID NO: 14 - protein, 11 aa, Artificial sequence
  Peptide component of the human protein MEMD/ALCAM
SSNTYTLTDVR

SEQ ID NO: 15 - protein, 582 aa, Homo sapiens
  Amino acid sequence of the human protein ALCAM/MEMD which is the antigen recognized by the antibody CR2407
ESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

(19) United States
(12) Patent Application Publication
(10) Pub. No.: US 2007/0287163 A1
(43) Pub. Date: Dec. 13, 2007

(54) Methods for Diagnosis of Acute Myeloid Leukemia

(75) Inventors: Cecilia Anna Wilhelmina Geuijen, Moerkapelle (NL); Cornelis Adriaan De Kruif, De Bilt (NL)

Correspondence Address: TRASK BRITT, P.O. BOX 2550, SALT LAKE CITY, UT, 84110, US

(73) Assignee: CRUCELL HOLLAND B.V., LEIDEN (NL)

(21) Appl. No.: 11/547,993

(22) PCT Filed: May 17, 2004
(86) PCT No.: PCT/EP04/50826

§ 371 Date: Oct. 9, 2006

(51) Int. Cl. G01N 33/574; A61K 48/00; A61P 35/02; C07H 21/04; C07K 14/00; C12N 15/00; C12N 5/06

(52) U.S. Cl. 435/7.23; 435/320.1; 435/366; 514/2; 530/350; 536/23.1

(57) ABSTRACT

The present invention provides a new marker for AML, binding molecules that specifically bind to the new marker, nucleic acid molecules encoding the binding molecules and compositions comprising the binding molecules. The binding molecules capable of specifically binding to the new marker can be used in the diagnosis of AML.

### FIELD OF THE INVENTION

The present invention relates to the field of medicine. The invention in particular relates to the identification of a new marker for acute myeloid leukemia (AML) and binding molecules capable of specifically binding to the new marker. The binding molecules are particular useful in the diagnosis and detection of AML.

### BACKGROUND OF THE INVENTION

Cancer describes a class of disorders and diseases characterised by the uncontrolled growth of aberrant cells. Currently, cancer is one of the most deadly diseases with about 1.2 million new cases of cancer being diagnosed each year in the United States of America alone.

One form of cancer, accounting for about 3% of all cancers in the United States of America, is leukemia. This malignant disease is characterised by an abnormal proliferation of white blood cells which can be detected in the peripheral blood and/or bone marrow. Leukemia can be broadly classified into acute and chronic leukemia. Further classification within these groups is essential as a precise diagnosis is necessary in order to determine prognosis and guide the choice of treatment. The acute leukemias can be subclassified into myeloid and lymphoid leukemias in a variety of ways, including cell morphology and cytochemistry. Despite the obvious morphological differences between the myeloid and lymphoid leukemias, immunophenotyping and cytogenetic analysis have become increasingly important in recent years to confirm and/or supplement diagnosis of leukemias.

Acute myeloid leukemia (AML) is the most common form of leukemia accounting for about 50% of all leukemia cases and even 85% of all acute leukemia cases involving adults. As a general criterion for the classification as AML, the cytoplasmic antigen myeloperoxidase should be demonstrated in the abnormal cell population either cytochemically or immunologically. Furthermore, the diagnosis of AML is supported by the expression of lineage-associated markers. Only a limited number of markers, such as inter alia CD13 and CD33, commonly expressed by a large number of AML-subtypes have been identified. Clearly, there is a continued need for more AML-specific markers that are associated with malignant cells from most patients with AML.

The present invention addresses this need by providing a new marker useful in the diagnosis/detection of a large number of AML-subtypes. The marker can supplement and improve present leukemia classification.

### DESCRIPTION OF THE FIGURES

FIG. 1 shows binding of the phage antibody SC02-407 (bold line) and a control phage antibody (SC02-428; normal line) to a set of five different primary AML blasts (FAB subtypes: AML 11, FAB-M0; AML3, FAB-M1; AML98028, FAB-M2; AML97028, FAB-M3; AML98081, FAB-M4).

FIG. 2A shows binding of the phage antibody SC02-407 (bold line) and a control phage antibody (SC02-428; normal line) to subpopulations of peripheral blood cells. FIG. 2B shows binding of the phage antibody SC02-407 (bold line) and a control phage antibody (SC02-428; normal line) to subpopulations of peripheral blood leukocytes.

FIG. 3 shows the binding intensity (depicted in mean fluorescence) of the phage antibody SC02-407 to AML in relation to the binding intensity of the phage antibody to the different cell populations in peripheral blood of a healthy donor.

FIG. 4 shows the expression vector pSyn-C03-HCgammal.

In FIG. 5 the expression construct pgG102-407C03 is shown.

In FIG. 6 the expression construct pSyn-C04-vl3 is shown.

FIG. 7 shows a Western blot of a HEp-2 cell lysate immunoprecipitated with a negative control IgG1 (CR2428; left lane), a positive control IgG1 directed against CD46 (CR2300; middle lane), or an anti-ALCAM IgG1 (CR2407; right lane). On the left side of the blot molecular weight markers are indicated.

FIG. 8 shows an immunoblot using an anti-ALCAM antibody. Two purified fractions derived from different affinity purifications and a control lysate where detected with the anti-ALCAM antibody. On the left side of the blot molecular weight markers are indicated.

### SUMMARY OF THE INVENTION

In the present invention a new marker for AML has been identified. Furthermore, a binding molecule capable of binding to the new marker has been identified and obtained by using phage display technology. Furthermore, the use of the new marker and the binding molecule binding to this marker in diagnosis and detection of AML has been described. The marker may also be useful in the prevention and/or treatment of AML.

### DETAILED DESCRIPTION OF THE INVENTION

The present invention pertains to a new marker for AML and binding molecules capable of binding this marker. These binding molecules can be used to target AML cells, e.g., for labeling. For such use, the binding molecules can be linked to a molecule that can be detected, in other words the binding molecule can have the form of an immunoconjugate. The invention provides methods for localizing AML cells ex vivo or in vitro or in vivo. This may be particularly useful for diagnostic or scientific purposes.

As a first aspect the invention provides a method of investigating the likelyhood that an individual suffers from AML. The method comprises the steps of contacting a sample comprising a heterogeneous cell population such as peripheral blood cells or bone marrow cells of the individual with a diagnostically effective amount of a binding molecule capable of binding to activated leukocyte cell adhesion molecule (ALCAM), and determining whether the binding molecule specifically binds to cells in the sample. Binding of the binding molecule to cells in the sample is indicative of AML. The sample may be purified before diagnosis by methods known to the skilled person. Peripheral blood may be purified by inter alia density gradient separation on Ficoll to obtain a sample comprising leukocytes. As used herein the term “acute myeloid leukemia” is characterized by an uncontrolled proliferation of progenitor cells of myeloid origin including, but not limited to, myeloid progenitor cells, myelomonocytic progenitor cells, and immature megakaryoblasts. AML can be classified into subtypes according to the FAB classification, i.e. FAB-M0, FAB-M1, FAB-M2, FAB-M3, FAB-M4, FAB-M5, FAB-M6 and FAB-M7. Preferably, the binding molecule capable of binding to activated leukocyte cell adhesion molecule (ALCAM) is an immunoconjugate, i.e. further comprises at least one tag, i.e. a detectable marker, such as a radionuclide or a fluorescent molecule, as described in more detail below.

ALCAM, also known as CD166, is a type I transmembrane protein and member of the immunoglobulin supergene family. It has a short cytoplasmic tail and an extracellular portion with five Ig domains, two N-terminal variable type domains followed by three constant type Ig domains (see Ohneda et al. (2001)). ALCAM is expressed by activated leukocytes, neurons, human TE cells, fibroblasts and epithelial cells. It is well known that ALCAM is a ligand of CD6. CD6/ALCAM interactions are thought to play an important role in regulating T-cell development and activation.

Recently, it has been shown that metastasizing malignant melanoma cells express ALCAM. In view thereof anti-ALCAM antibodies have been used as a diagnostic marker of primary malignant melanoma tumor progression (see Van Kempen et al. (2000)). Moreover, ALCAM has been shown to be present on a variety of both primary and metastatic human cancers (other than metastatic malignant melanoma), such as ovarian, lung, prostate, pancreatic, colon, and breast cancer cells, and that anti-ALCAM antibodies may be used to detect such cancers (see US 2004/0048319 and WO 03/093443). ALCAM has not been implicated in cancers of hematopoietic origin including leukemias such as AML.

In a preferred embodiment the method of the invention is performed by flow cytometry. As used herein, the term “flow cytometry” has its art recognized meaning which generally refers to a technique for characterizing biological particles, such as whole cells or cellular constituents, by flow cytometry. Methods for performing flow cytometry on samples of immune cells are well known in the art (see e.g., Jaroszeski et al., Method in Molecular Biology, (1998), vol. 91: Flow Cytometry Protocols, Hummama Press; Longobanti Givan, (1992) Flow Cytometry, First Principles, Wiley Liss). All known forms of flow cytometry are intended to be included, particularly fluorescence activated cell sorting (FACS), in which fluorescent labeled molecules are evaluated by flow cytometry.

Any technique may be employed in flow cytometry which is not unduly detrimental to the viability of the selected cells. A preferred apparatus for performing flow cytometry in the method of the invention is a fluorescence activated cell sorter (FACS). A further aspect of the invention is therefore a method of diagnosing AML, wherein ALCAM cell surface expression of AML cells is determined using a fluorescence activated cell sorter. Fluorescence activated cell sorters can have varying degrees of sophistication, such as multiple color channels, low angle and obtuse light scattering detecting channels, impedance channels, etc. Cells may be selected against dead cells by employing dyes associated with dead cells (e.g. propidium iodide). The FACS apparatus commonly includes a light source, usually a laser, and several detectors for the detection of cell particles or subpopulations of cells in a mixture using light scatter or light emission parameters. The underlying mechanisms of FACS are well known in the art, and essentially involve scanning (e.g., counting, sorting by size or fluorescent label) single particles are they flow in a liquid medium past an excitation light source. Light is scattered and fluorescence is emitted as light from the excitation source strikes the moving particle. Forward scatter (FSC, light scattered in the forward direction, i.e., the same direction as the beam) provides basic morphological information about the particles, such as cell size and morphology. Light that is scattered at 90° to the incident beam is due to refracted or reflected light, and is referred to as side angle scatter (SSC). This parameter measures the granularity and cell surface topology of the particles. Collectively, scatter signals in both the forward and wide angle direction are used to identify subpopulations of cells based on cell size, morphology and granularity. This information is used to distinguish various cellular populations in a heterogeneous sample. Characterization of leukemias by immunophenotyping via flow cytometry is particularly helpful when the morphology is difficult to interpret such as for instance in the classification of acute leukemia (for a review on flow cytometric analysis of acute leukemia see Jennings and Foon (1997), which is incorporated by reference herein). In most cases, immunophenotyping can distinguish between lymphoblastic (ALL) and myeloblastic (AML) forms. This distinction is frequently difficult by routine morphological criteria and the two forms require different therapies and have different prognoses. Besides that, in patients with AML, immunophenotyping can also be useful in detecting certain relatively uncommon subsets, such as acute megakaryocytic leukemia (FAB-M7) and acute erythroleukemia (FAB-M6).

The invention also provides methods of aiding diagnosis of AML in an individual using the binding molecule of the invention, i.e. the binding molecule that binds to ALCAM. As used herein, methods for “aiding diagnosis” means that these methods assist in making a clinical determination and may or may not be conclusive with respect to the definitive diagnosis. Preferably, additional methods that can be used to determine malignant cells such as AML cells are performed in the diagnosis. In yet a further aspect the invention is directed to a method according to the invention wherein the method further comprises the steps of identifying malignant cells in the sample before, during or after the step of contacting a sample comprising peripheral blood cells or bone marrow cells of an individual with a diagnostically effective amount of a binding molecule capable of binding to activated leukocyte cell adhesion molecule (ALCAM), and determining whether the binding molecule specifically binds to a malignant cell. Binding is herein indicative of AML. The sample can be a heterogeneous cell population comprising among others malignant cells. Preferably, the blast cell populations are detected in the cell population. Malignant cells can be identified by means of inter alia morphology and histochemistry, cytogenetic analysis and selected molecular studies. In patients with for instance acute leukemia special histochemical stains can be performed on blood or bone marrow specimens to confirm the diagnosis. The stains most commonly used include myeloperoxidase, PAS, Sudan Black B, and esterase. Multiparameter flow cytometry measurements using marker molecules also allow for the differential analysis of normal and abnormal/malignant cells within a peripheral blood or bone marrow specimen of patients suffering from leukemia such as acute leukemia. Identification of the malignant cells by flow cytometry can be performed with at least one other binding molecule. Preferably, this other binding molecule binds to an antigen other than ALCAM. The antigen can be associated with non-malignant cells, but preferably is associated with malignant cells. In another embodiment the antigen is specific for a subset of malignant cells such as an antigen associated with acute leukemia cells such as AML cells. In yet another embodiment the antigen is specific for subtypes within the specific subset, such as an AML-antigen specific for one or more subtypes according to the FAB classification. In a further embodiment a combination of several binding molecules each binding molecule binding a different antigen specific for a malignant leukemic cell such as an AML cell may be used to identify the malignant cell population. The different binding molecules may be used simultaneously or independent from each other. Examples of suitable antigens expressed by AML cells include, but are definitely not limited to, antiglycophorin A, CD11B, CD13, CD14, CD15, CD33, CD34, CD36, CD38, CDw4l, CD45, CD61, CD71, CD117, CD133, and HLA-DR. The common phenotype of the different AML FAB-subtypes is shown in Table 1 of Jennings and Foon (1997) (the complete document and specifically Table 1 are incorporated by reference herein). Antigens on AML cells should be used in leukemia diagnosis, along with a battery of lineage-specific T- and B-lymphocyte markers to help distinguish AML from ALL and mixed-lineage or biphenotypic or biclonal leukemias. Lineage-associated B-lymphocytic antigens CD10, CD19, CD20, CD22, and CD24 may be present in 10% to 20% of AMLs; similarly, CD2, CD3, CD5, and CD7 lineage-specific T-lymphocytic antigens are present in 20% to 40% of AMLs. The expression of lymphoid-associated antigens by AML cells is relatively common but has no prognostic significance. Table 2 of Jennings and Foon (1997) which is incorporated by reference herein describes the common phenotype of lymphoblastic leukemias such as B-precursor ALL, Pre-B ALL, B-ALL and T-ALL. Tables 3 and 4 of Jennings and Foon (1997), which are also incorporated by reference herein, describes the common phenotype of B- and T-lymphoproliferative disorders, respectively.

Detection of residual AML tumor cells during or immediately following therapy could indicate that the treatment protocol is incomplete or that the tumor cells are developing resistance to the therapy. At the time of leukemia remission, the level of leukemia cells in the body is largely undetectable by current morphologic and cytochemical methods. The methods as described herein can be useful in the detection of minimal residual disease or the evaluation of tumor response to therapy and might lead to more accurate determinations and could improve clinical management by providing measures by which to guide therapeutic intervention and thereby result in increased cure rates.

Preferably, the binding molecules capable of specifically binding to ALCAM are labeled with a detectable label such as a fluorescent molecule. More preferably, the other binding molecule is also labeled with a detectable label such as a fluorescent molecule. Most preferably, the labels of the binding molecules of the invention and the other binding molecules are different.

Preferably, the malignant cells are identified by flow cytometry. Malignant cells, such as AML cells, within blood, bone marrow, other hematopoietic or other non-hematopoietic compartments of the body can be selected based on their specific pattern in for instance FACS analysis.

The individual which is subjected to diagnosis is selected from the group consisting of an individual suspected of having AML, an individual having AML, an individual recovered from AML, and an individual relapsed from AML.

In a preferred embodiment the binding molecule as used in the present invention is a human binding molecule capable of specifically binding ALCAM. Binding molecules as meant herein can be intact immunoglobulin molecules such as polyclonal or monoclonal antibodies, in particular human monoclonal antibodies, or the binding molecules can be antigen-binding fragments including, but not limited to, Fab, F(ab′), F(ab′)2, Fv, dAb, Fd, complementarity determining region (CDR) fragments, single-chain antibodies (scFv), bivalent single-chain antibodies, diabodies, triabodies, tetrabodies, and (poly)peptides that contain at least a fragment of an immunoglobulin that is sufficient to confer specific antigen binding to the (poly)peptides, e.g. chimeric or humanized antibodies. The above fragments may be produced synthetically or by enzymatic or chemical cleavage of intact immunoglobulins or they may be genetically engineered by recombinant DNA techniques. The methods of production are well known in the art and are described, for example, in Antibodies: A Laboratory Manual, Edited by: E. Harlow and D, Lane (1988), Cold Spring Harbor Laboratory, Cold Spring Harbor, N.Y., which is incorporated herein by reference. The binding molecules of the invention can be used in non-isolated or isolated form. Binding molecules include the immunoglobulin classes and subclasses known in the art: IgA, IgD, IgE, IgG, and IgM, and several of these may be further divided into subclasses (isotypes), e.g., IgA1, IgA2, IgG1, IgG2, IgG3 and IgG4. Typically, binding molecules according to the invention can bind to ALCAM with an affinity constant (Kd-value) that is lower than 0.2*10⁻⁴ M, 1.0*10⁻⁵ M, 1.0*10⁻⁶ M, 1.0*1 0⁻⁷ M, preferably lower than 1.0*10⁻⁸ M, more preferably lower than 1.0*10⁻⁹ M, more preferably lower than 1.0*10⁻¹⁰ M, even more preferably lower than 1.0*10⁻¹¹ M, and in particular lower than 1.0*10⁻¹² M. The affinity constants can vary for antibody isotypes. For example, affinity binding for an IgM isotype refers to a binding affinity of at least about 1.0*10⁻⁷ M. Affinity constants can be measured using surface plasmon resonance, i.e. an optical phenomenon that allows for the analysis of real-time biospecific interactions by detection of alterations in protein concentrations within a biosensor matrix, for example using the BIAcore system (Pharmacia Biosensor AB, Uppsala, Sweden).

The binding molecules according to the invention may bind to ALCAM in soluble form or may bind to ALCAM bound or attached to a carrier or substrate, e.g., microtiter plates, membranes and beads, etc. Carriers or substrates may be made of glass, plastic (e.g., polystyrene), polysaccharides, nylon, nitrocellulose, or teflon, etc. The surface of such supports may be solid or porous and of any convenient shape. Furthermore, the binding molecules may bind to ALCAM in purified or non-purified form and/or in isolated or non-isolated form. Preferably, the binding molecules are capable of specifically binding to ALCAM associated with cells, such as ALCAM positive cells, e.g. AML cells or cells transfected with ALCAM, or portions or parts of these cells comprising ALCAM or a fragment thereof.

A functional variant of a binding molecule or fragment thereof is another aspect of the invention. Functional variants include, but are not limited to, derivatives that are substantially similar in primary structural sequence, but which contain e.g. in vitro or in vivo modifications, chemical and/or biochemical, that are not found in the parent binding molecule. Such modifications are well known to the skilled person and include inter alia acetylation, acylation, covalent attachment of a lipid or lipid derivative, disulfide bond formation, glycosylation, methylation, pegylation, phosphorylation, prenylation, and the like.

Alternatively, a functional variant refers to a binding molecule or fragment thereof that comprises a nucleotide and/or amino acid sequence that is altered by one or more nucleotides and/or amino acids compared to the nucleotide and/or amino acid sequences of the parent binding molecule and that is still capable of competing for binding to the binding partner, e.g. human ALCAM, with the parent binding molecule. In other words, the modifications in the amino acid and/or nucleotide sequence of the parent binding molecule do not significantly affect or alter the binding characteristics of the binding molecule encoded by the nucleotide sequence or containing the amino acid sequence, i.e. the binding molecule is still able to recognize and bind its target. The functional variant may have conservative sequence modifications including nucleotide and amino acid substitutions, additions and deletions. Functional variants can be obtained by altering the parent binding molecules or parts thereof by general molecular biology methods known in the art including, but not limited to, error-prone PCR, oligonucleotide-directed mutagenesis, random PCR-mediated mutagenesis, and site-directed mutagenesis. Furthermore, functional variants can comprise truncations of the amino acid sequence at either or both the amino or carboxy termini. Guidance in determining which amino acid residues may be substituted, inserted, or deleted without abolishing immunological activity may be found using computer programs well known in the art. A mutation in a nucleotide sequence can be a single alteration made at a locus (a point mutation), such as transition or transversion mutations, or alternatively, multiple nucleotides may be inserted, deleted or changed at a single locus. In addition, one or more alterations may be made at any number of loci within a nucleotide sequence. The mutations may be performed by any suitable method known in the art.

In a preferred embodiment the human binding molecule according to the invention is characterized in that it comprises at least a heavy chain CDR3 region comprising the amino acid sequence of SEQ ID NO:3.

In a more preferred embodiment the human binding molecule according to the invention is characterized in that it comprises a heavy chain comprising the amino acid sequence of SEQ ID NO:11 (with the proviso that the signal peptide of amino acid 1-21 is absent).

In an even more preferred embodiment, the human binding molecule according to the invention comprises a heavy chain comprising the amino acid sequence shown in SEQ ID NO:11 (with the proviso that the signal peptide of amino acid 1-21 is absent) and a light chain comprising the amino acid sequence of SEQ ID NO:13 (with the proviso that the signal peptide of amino acid 1-21 is absent).

In yet a further aspect, the invention includes immunoconjugates, i.e. molecules comprising at least one binding molecule as defined herein and further comprising at least one tag, preferably a detectable molecule. Also contemplated in the present invention are mixtures of immunoconjugates according to the invention or mixtures of at least one immunoconjugates according to the invention and another molecule, such as a diagnostic agent or another binding molecule or immunoconjugate. In a further embodiment, the immunoconjugates of the invention may comprise more than one tag. These tags can be the same or distinct from each other and can be joined/conjugated non-covalently to the binding molecules. The tags can also be joined/conjugated directly to the binding molecules through covalent bonding, including, but not limited to, disulfide bonding, hydrogen bonding, electrostatic bonding, recombinant fusion and conformational bonding. Alternatively, the tags can be joined/conjugated to the binding molecules by means of one or more linking compounds. Techniques for conjugating tags to binding molecules, are well known, see, e.g., Arnon et al., Monoclonal Antibodies For Immunotargeting Of Drugs In Cancer Therapy, p. 243-256 in Monoclonal Antibodies And Cancer Therapy (1985), Edited by: Reisfeld et al., A. R. Liss, Inc.; Hellstrom et al., Antibodies For Drug Delivery, p. 623-653 in Controlled Drug Delivery, 2nd edition (1987), Edited by: Robinson et al., Marcel Dekker, Inc.; Thorpe, Antibody Carriers Of Cytotoxic Agents, p. 475-506 In Cancer Therapy: A Review, in Monoclonal Antibodies'84 : Biological And Clinical Applications (1985), Edited by: Pinchera et al.; Analysis, Results, And Future Prospective Of The Therapeutic Use Of Radiolabeled Antibody In Cancer Therapy, p. 303-316 in Monoclonal Antibodies For Cancer Detection And Therapy (1985), Edited by: Baldwin et al., Academic Press.

The binding molecules as described in the present invention can be conjugated to tags and be used for detection and/or analytical and/or diagnostic purposes. The tags used to label the binding molecules for those purposes depend on the specific detection/analysis/diagnosis techniques and/or methods used such as inter alia immunohistochemical staining of tissue samples, flow cytometric detection, scanning laser cytometric detection, fluorescent immunoassays, enzyme-linked immunosorbent assays (ELISA's), radioimmunoassays (RIA's), bioassays (e.g., growth inhibition assays, migration assays), Western blotting applications, immunoprecipitations, immunofluorescence, enzyme immunoassay (EIA), immunopurification, etc. The binding molecule can be directly labeled by coupling (i.e. physically linking) a detectable tag to the binding molecule. Alternatively, the tag or binding molecule can be indirectly labeled by coupling the tag or binding molecule with a substance which reacts with a detectable substance.

For immunohistochemical staining of samples preferred labels are enzymes that catalyze production and local deposition of a detectable product. Enzymes typically conjugated to binding molecules to permit their immunohistochemical visualization as well as (luminescent) substrates for production and deposition of visually detectable products are well-known in the art. The binding molecules of the immunoconjugate of the invention can also be labeled using colloidal gold or they can be labeled with radioisotopes, such as ³³p, ³²p ³⁵S, ³H, and ¹²⁵I. The binding molecules of the invention may be conjugated to photoactive agents or dyes such as fluorescent and other chromogens or dyes to use the so obtained immunoconjugates in photoradiation, phototherapy, or photodynamic therapy.

Preferably, the binding molecules of the present invention are used for flow cytometric detections, scanning laser cytometric detections, or fluorescent immunoassays. In these ways of detection they can usefully be labeled with fluorescent molecules such as fluorophores. A wide variety of fluorophores useful for fluorescently labeling the binding molecules of the present invention include, but are not limited to, Alexa Fluor and Alexa Fluor&commat dyes, BODIPY dyes, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Cy2, Cy3, Cy3.5, Cy5, Cy5.5, Cy7, fluorescein isothiocyanate (FITC), allophycocyanin (APC), R-phycoerythrin (PE), peridinin chlorophyll protein (PerCP), Texas Red, fluorescence resonance energy tandem fluorophores such as PerCP-Cy5.5, PE-Cy5, PE-Cy5.5, PE-Cy7, PE-Texas Red, and APC-Cy7. When the binding molecules of the present invention are used for secondary detection using labeled avidin, streptavidin, captavidin or neutravidin, the binding molecules may be labeled with biotin.

Furthermore, the binding molecules or immunoconjugates of the invention can also be attached to solid supports, which are particularly useful for immunoassays or purification of the binding partner. Such solid supports might be porous or nonporous, planar or nonplanar and include, but are not limited to, glass, cellulose, polyacrylamide, nylon, polystyrene, polyvinyl chloride or polypropylene supports. The binding molecules can also for example usefully be conjugated to filtration media, such as NHS-activated Sepharose or CNBr-activated Sepharose for purposes of immunoaffinity chromatography. They can also usefully be attached to paramagnetic microspheres, typically by biotin-streptavidin interaction. Preferably, the microspheres can be used for isolation of cells that express or display human ALCAM, such as AML cells. As another example, the binding molecules of the present invention can usefully be attached to the surface of a microtiter plate for ELISA.

In yet a further aspect, the invention provides a method for selecting ALCAM presenting cells comprising the steps of (a) contacting a sample comprising ALCAM presenting cells with a binding molecule of the invention capable of specifically binding to ALCAM, (b) separating the cells that bind to the binding molecule from the cells that do not bind to the binding molecule, and (c) recovering the cells which are bound by the binding molecule. In a specific embodiment the method comprises the further step of selecting malignant cells from the sample. This step may be performed before, during or after contacting a sample comprising ALCAM presenting cells with a binding molecule of the invention capable of specifically binding to ALCAM. The ALCAM presenting cells can be AML cells. The step of separating the cells that bind to the binding molecule from the cells that do not bind to the binding molecule can inter alla be performed by affinity separation to provide a substantially pure population of cells. Techniques for affinity separation may include, flow cytometry, magnetic separation, using binding molecule-coated magnetic beads, affinity chromatography, cytotoxic agents joined to a monoclonal antibody or used in conjunction with a monoclonal antibody, e.g. complement and cytotoxins, and “panning” with binding molecules attached to a solid matrix, e.g. plate, or other convenient technique.

In a further aspect, the invention provides compositions comprising at least one binding molecule, functional variant thereof, fragment thereof, or immunoconjugate according to the invention or a combination thereof. In addition to that, the compositions may comprise inter alia stabilising molecules, such as albumin or polyethylene glycol, or salts.

The above mentioned molecules or compositions may be employed in conjunction with other molecules useful in diagnosis. They can be used in vitro, ex vivo or in vivo. The molecules are typically formulated in a diagnostically effective amount. Dosage regimens can be adjusted to provide the optimum desired response (e.g., a diagnostic response).

In another aspect the binding molecules, preferably the human binding molecules such as human monoclonal antibodies according to the invention, the functional variants or fragments thereof, the immunoconjugates according to the invention, the nucleic acid molecules according to the invention, the compositions according to the invention can be used as medicaments, i.e. in the treatment of AML. In the treatment of AML the binding molecules of the invention may be used as pharmaceutical compositions comprising at least one binding molecule according to the invention, at least one functional variant or fragment thereof, at least one immunoconjugate according to the invention, at least one composition according to the invention, or combinations thereof. The pharmaceutical composition of the invention further comprises at least one pharmaceutically acceptable excipient. Furthermore, the binding molecules can be used in combination, e.g., as a pharmaceutical composition comprising two or more binding molecules or fragments thereof. The mixture may further comprise at least one other therapeutic agent.

The choice of the optimal route of administration of the pharmaceutical compositions, such as oral or parenteral administration, will be influenced by several factors including the physico-chemical properties of the active molecules within the compositions, the urgency of the clinical situation and the relationship of the plasma concentrations of the active molecules to the desired therapeutic effect. Routes of administration are well known to persons skilled in the art.

The binding molecules of the present invention can also be used to modulate, either by enhancing or reducing, the association of ALCAM with a ligand such as inter alia CD6. The association of ALCAM with a ligand can be impacted by contacting an ALCAM molecule present on a cell surface, with an agent such as a binding molecule that modulates the binding of the ligand to ALCAM.

In case immunoconjugates according to the invention are used in the treatment of AML useful tags may include, but are not limited to, toxic substances, radioactive substances, liposomes, enzymes, polynucleotide sequences, plasmids, proteins, peptides or combinations thereof. Toxic substances include, but are not limited to, cytotoxic agents, such as small molecule toxins or chemotherapeutic agents, or enzymatically active toxins of bacterial, fungal, plant or animal origin, or fragments thereof. In general, suitable chemotherapeutic agents are described in Remington's Pharmaceutical Sciences, 18th edition (1990), Edited by: A. R. Gennaro, Mack Publishing Co., Philadelphia and in Goodman and Gilman's The Pharmacological Basis of Therapeutics, 7th edition (1985), Edited by: A. G. Gilman, L. S. Goodman, T. W. Rall and F. Murad. MacMillan Publishing Co., New York. Suitable chemotherapeutic agents that are still in the experimental phase are known to those of skill in the art and might also be used as toxic substances in the present invention. Immunoconjugates comprising enzymes may be useful in antibody-directed enzyme-prodrug therapy (ADEPT). In this technique enzymes are conjugated to binding molecules. This conjugation converts the enzymes into inactive prodrugs. The binding molecule-enzyme conjugates are then administered and bind to the binding partner of the binding molecule. After clearance of the conjugates from the circulation, prodrugs are administered, which are converted into active drugs by the enzyme of the conjugates. Passive uptake of the active drugs into the target cells will then occur. Also contemplated within the present invention are binding molecules of the immunoconjugate of the invention that are labeled with radionuclides. Suitable radionuclides include, but are not limited to, radionuclides that emit alpha radiation; radionuclides that emit beta radiation; radionuclides that emit gamma radiation; Auger-electron-emitting radionuclides, and other radiolabeled halogens. The skilled man will appreciate that other suitable radionuclides can also be identified as suitable in the present invention. The choice of radionuclide will be dependent on many factors such as, e.g., the stage of the disease to be treated, the patient to be treated and the like. Binding molecules can be attached to radionuclides directly or indirectly via a chelating agent by methods well known in the art. In another embodiment, the binding molecules of the immunoconjugate of the invention can be conjugated to liposomes to produce so-called immunoliposomes. In yet another embodiment, the binding molecules of the invention may be linked to water-soluble, biodegradable polymers, such as for instance polymers of hydroxypropylmethacrylamine (HPMA). The polymers have toxic substances linked on separate sites of the polymers with the use of appropriate degradable spacers to allow for release of the toxic substances. The above described polymers are also called immunopolymers. In another aspect the binding molecules of the invention may be conjugated/attached to one or more antigens. Preferably, these antigens are antigens which are recognised by the immune system of a subject to which the binding molecule-antigen conjugate is administered. The binding molecules will bind to the AML cells comprising human ALCAM and the antigens attached to the binding molecules will initiate a powerful T-cell attack on the conjugate which will eventually lead to the destruction of the cell.

A nucleic acid molecule encoding a (human) binding molecule according to the invention is also a part of the present invention. Nucleic acid molecule refers to a polymeric form of nucleotides and includes both sense and antisense strands of RNA, cDNA, genomic DNA, and synthetic forms and mixed polymers of the above. A nucleotide refers to a ribonucleotide, deoxynucleotide or a modified form of either type of nucleotide. The term also includes single- and double-stranded forms of DNA. In addition, a polynucleotide may include either or both naturally-occurring and modified nucleotides linked together by naturally-occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages, charged linkages, pendent moieties, intercalators, chelators, alkylators, and modified linkages. Nucleic acid molecule is also intended to include any topological conformation, including single-stranded, double-stranded, partially duplexed, triplex, hairpinned, circular and padlocked conformations. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. A reference to a nucleic acid sequence encompasses its complement unless otherwise specified. Thus, a reference to a nucleic acid molecule having a particular sequence should be understood to encompass its complementary strand, with its complementary sequence. The complementary strand is also useful, e.g., for antisense therapy, hybridization probes and PCR primers. In a preferred embodiment, the nucleic acid molecules are isolated or purified.

The skilled man will appreciate that functional variants of the nucleic acid molecules of the invention are also intended to be a part of the present invention. Functional variants are nucleic acid sequences that can be directly translated, using the standard genetic code, to provide an amino acid sequence identical to that translated from the parent nucleic acid molecules. Preferably, the nucleic acid molecules encode binding molecules comprising a CDR3 region, preferably a heavy chain CDR3 region, comprising the amino acid sequence of SEQ ID NO:3. Even more preferably, the nucleic acid molecules encode binding molecules comprising a heavy chain comprising the amino acid sequence of SEQ ID NO:11 (without the signal peptide of amino acids 1-21). In yet another embodiment, the nucleic acid molecules encode binding molecules comprising a heavy chain comprising the amino acid sequence of SEQ ID NO:11 (without the signal peptide of amino acids 1-21) and a light chain comprising the amino acid sequence of SEQ ID NO:13 (without the signal peptide of amino acids 1-21).

In a further aspect, the invention provides compositions comprising at least one nucleic acid molecule as defined in the present invention. The compositions may comprise aqueous solutions such as aqueous solutions containing salts (e.g., NaCl or salts as described above), detergents (e.g., SDS) and/or other suitable components.

In yet a further aspect, the invention relates to a vector comprising at least one nucleic acid molecule according to the invention. The term “vector” denotes a nucleic acid molecule into which a second nucleic acid molecule can be inserted for introduction into a host where it will be replicated, and in some cases expressed. In other words, a vector is capable of carrying a second nucleic acid molecule to which it has been linked. Cloning as well as expression vectors are contemplated by the term “vector”, as used herein. Vectors include, but are not limited to, plasmids, cosmids, bacterial artificial chromosomes (BAC) and yeast artificial chromosomes (YAC) and vectors derived from bacteriophages or plant or animal (including human) viruses. Vectors comprise an origin of replication recognised by the proposed host and in case of expression vectors, promoter and other regulatory regions recognised by the host. A vector containing a second nucleic acid molecule is introduced into a cell by electroporation, transformation, transfection, or by making use of viral entry mechanisms. Certain vectors are capable of autonomous replication in a host into which they are introduced (e.g., bacterial vectors having a bacterial origin of replication). Other vectors can be integrated into the genome of a host upon introduction into the host, and thereby are replicated along with the host genome. Vectors can be used for cloning and/or for expression of the binding molecules of the invention and might even be used for gene therapy purposes. Vectors comprising one or more nucleic acid molecules according to the invention operably linked to one or more expression-regulating nucleic acid molecules are also covered by the present invention. The choice of the vector is dependent on the recombinant procedures followed and the host used. Introduction of vectors in host cells can be effected by inter alia calcium phosphate transfection, virus infection, DEAE-dextran mediated transfection, lipofectamin transfection or electroporation. Preferably, the vectors contain one or more selection markers. The choice of the markers may depend on the host cells of choice, although this is not critical to the invention as is well known to persons skilled in the art. Vectors comprising one or more nucleic acid molecules encoding the binding molecules as described above operably linked to one or more nucleic acid molecules encoding proteins or peptides that can be used to isolate the binding molecules are also covered by the invention.

Hosts containing one or more copies of the vectors mentioned above are an additional subject of the present invention. Preferably, the hosts are host cells. Host cells include, but are not limited to, cells of mammalian, plant, insect, fungal or bacterial origin. Bacterial cells include, but are not limited to, cells from Gram positive bacteria such as several species of the genera Bacillus, Streptomyces and Staphylococcus or cells of Gram negative bacteria such as several species of the genera Escherichia and Pseudomonas. In the group of fungal cells preferably yeast cells are used. Expression in yeast can be achieved by using yeast strains such as inter alia Pichia pastoris, Saccharomyces cerevisiae and Hansenula polymorpha. Furthermore, insect cells such as cells from Drosophila and Sf9 can be used as host cells. Besides that, the host cells can be plant cells, such as for instance transformed (transgenic) plant cells. Additionally, a suitable expression system can be a baculovirus system. Expression systems using mammalian cells such as Chinese Hamster Ovary (CHO) cells, COS cells, BHK cells or Bowes melanoma cells are preferred in the present invention. Mammalian cells provide expressed proteins with posttranslational modifications that are most similar to natural molecules of mammalian origin. Even more preferably, the host cells are human cells. Examples of human cells are inter alia HeLa, 911, AT1080, A549, 293 and HEK293T cells. Preferred mammalian cells are human retina cells such as 911 cells or the cell line deposited at the European Collection of Cell Cultures (ECACC), CAMR, Salisbury, Wiltshire SP4 OJG, Great Britain on 29 Feb. 1996 under number 96022940 and marketed under the trademark PER.C6® (PER.C6 is a registered trademark of Crucell Holland B.V.). In preferred embodiments, the human producer cells comprise at least a functional part of a nucleic acid sequence encoding an adenovirus E1 region in expressible format. In even more preferred embodiments, said host cells are derived from a human retina and immortalised with nucleic acids comprising adenoviral E1 sequences, such as the cell line deposited at the European Collection of Cell Cultures (ECACC), CAMR, Salisbury, Wiltshire SP4 OJG, Great Britain on 29 Feb. 1996 under number 96022940 and marketed under the trademark PER.C6®, and derivatives thereof. Production of recombinant proteins in host cells can be performed according to methods well known in the art. For the purposes of this application “PER.C6” refers to cells deposited under number 96022940 or ancestors, passages up-stream or downstream as well as descendants from ancestors of deposited cells, as well as derivatives of any of the foregoing. The use of the cells marketed under the trademark PER.C6® as a production platform for proteins of interest has been described in WO 00/63403 the disclosure of which is incorporated herein by reference in its entirety. The binding molecules of the invention can be produced by a method comprising the following steps: a) culturing a host as described above under conditions conducive to the expression of the binding molecules, and b) optionally, recovering the expressed binding molecules. The expressed binding molecules can be recovered from the cell free extract, but preferably they are recovered from the culture medium. Methods to recover proteins, such as binding molecules, from cell free extracts or culture medium are well known to the man skilled in the art. Binding molecules as obtainable by the above described method are also a part of the present invention.

Alternatively, next to the expression in hosts, such as host cells, the binding molecules of the invention can be produced synthetically by conventional peptide synthesizers or in cell-free translation systems using RNA's derived from DNA molecules according to the invention. Binding molecule as obtainable by the above described synthetic production methods or cell-free translation systems are also a part of the present invention. In yet another alternative embodiment, binding molecules according to the present invention, preferably human binding molecules specifically binding to ALCAM or fragments thereof, may be generated by transgenic non-human mammals, such as for instance transgenic mice or rabbits, that express human immunoglobulin genes. Protocols for immunizing non-human mammals are well established in the art. See Using Antibodies: A Laboratory Manual, Edited by: E. Harlow, D. Lane (1998), Cold Spring Harbor Laboratory, Cold Spring Harbor, N.Y. and Current Protocols in Immunology, Edited by: J. E. Coligan, A. M. Kruisbeek, D. H. Margulies, E. M. Shevach, W. Strober (2001), John Wiley & Sons Inc., New York, the disclosures of which are incorporated herein by reference.

The binding molecules of the invention can be identified a) contacting a phage library of binding molecules, preferably human binding molecules, with material comprising ALCAM or a part thereof, b) selecting at least once for a phage binding to the material comprising ALCAM or a part thereof, and c) separating and recovering the phage binding to the material comprising ALCAM or a part thereof. The material comprising ALCAM can be cells transfected with ALCAM expression plasmids, isolated human ALCAM, the extracellular part thereof, fusion proteins comprising such, and the like. Preferably, the material comprising ALCAM are AML cells. Phage display methods for identifying and obtaining binding molecules, e.g. antibodies, are by now well-established methods known by the person skilled in the art. They are e.g. described in U.S. Pat. No. 5,696,108; Burton and Barbas, 1994; and de Kruif et al., 1995b. For the construction of phage display libraries, collections of human monoclonal antibody heavy and light chain variable region genes are expressed on the surface of bacteriophage, preferably filamentous bacteriophage, particles, in for example single chain Fv (scFv) or in Fab format (see de Kruif et al., 1995b). Large libraries of antibody fragment-expressing phages typically contain more than 1.0*10⁹ antibody specificities and may be assembled from the immunoglobulin V regions expressed in the B-lymphocytes of immunized- or non-immunized individuals. Alternatively, phage display libraries may be constructed from immunoglobulin variable regions that have been partially assembled in vitro to introduce additional antibody diversity in the library (semi-synthetic libraries). For example, in vitro assembled variable regions contain stretches of synthetically produced, randomized or partially randomized DNA in those regions of the molecules that are important for antibody specificity, e.g. CDR regions. Antigen specific phage antibodies can be selected from the library by immobilising target antigens such as ALCAM or fragments thereof on a solid phase and subsequently exposing the target antigens to a phage library to allow binding of phages expressing antibody fragments specific for the solid phase-bound antigen. Non-bound phages are removed by washing and bound phages eluted from the solid phase for infection of Escherichia coli (E.coli) bacteria and subsequent propagation. Multiple rounds of selection and propagation are usually required to sufficiently enrich for phages binding specifically to the target antigen. Phages may also be selected for binding to complex antigens such as complex mixtures of proteins or whole cells such as cells transfected with human ALCAM expression plasmids or cells naturally expressing human ALCAM such as AML cells. Selection of antibodies on whole cells has the advantage that target antigens are presented in their native configuration, i.e. unperturbed by possible conformational changes that might have been introduced in the case where an antigen is immobilized to a solid phase. Antigen specific phage antibodies can be selected from the library by incubating a cell population of interest, expressing known and unknown antigens on their surface, with the phage antibody library to let for example the scFv or Fab part of the phage bind to the antigens on the cell surface. After incubation and several washes to remove unbound and loosely attached phages, the cells of interest are stained with specific fluorescent labeled antibodies and separated on a Fluorescent Activated Cell Sorter (FACS). Phages that have bound with their scFv or Fab part to these cells are eluted and used to infect Escherichia coli to allow amplification of the new specificity. Generally, one or more selection rounds are required to separate the phages of interest from the large excess of non-binding phages. Monoclonal phage preparations can be analyzed for their specific staining patterns and allowing identification of the antigen being recognized (De Kruif et al., 1995a). The phage display method can be extended and improved by subtracting non-relevant binders during screening by addition of an excess of non-target molecules that are similar, but not identical, to the target, and thereby strongly enhance the chance of finding relevant binding molecules (This process is referred to as the Mabstract® process. Mabstract® is a pending trademark application of Crucell Holland B. V., see also U.S. Pat. No. 6,265,150 which is incorporated herein by reference).

In yet a further aspect, the invention provides a method of obtaining a binding molecule, preferably a human binding molecule or a nucleic acid molecule according to the invention, wherein the method comprises the steps of a) performing the above described method of identifying binding molecules, preferably human binding molecules such as human monoclonal antibodies or fragments thereof according to the invention, or nucleic acid molecules according to the invention, and b) isolating from the recovered phage the human binding molecule and/or the nucleic acid encoding the human binding molecule. Once a new monoclonal phage antibody has been established or identified with the above mentioned method of identifying binding molecules or nucleic acid molecules encoding the binding molecules, the DNA encoding the scFv or Fab can be isolated from the bacteria or phages and combined with standard molecular biological techniques to make constructs encoding bivalent scFv's or complete human immunoglobulins of a desired specificity (e.g. IgG, IgA or IgM). These constructs can be transfected into suitable cell lines and complete human monoclonal antibodies can be produced (see Huls et al., 1999; Boel et al., 2000).

Kits comprising at least one binding molecule, preferably human binding molecule such as human monoclional antibody according to the invention, at least one variant or fragment thereof, at least one immunoconjugate according to the invention, at least one nucleic acid molecule according to the invention, at least one composition according to the invention, at least one pharmaceutical composition according to the invention, at least one vector according to the invention, at least one host according to the invention or a combination thereof are also a part of the present invention. Optionally, the above described components of the kits of the invention are packed in suitable containers and labeled for diagnosis and/or treatment of the indicated conditions. The above-mentioned components may be stored in unit or multi-dose containers, for example, sealed ampules, vials, bottles, syringes, and test tubes, as an aqueous, preferably sterile, solution or as a lyophilized, preferably sterile, formulation for reconstitution. The containers may further include other materials desirable from a commercial and user standpoint, including other buffers, diluents, filters, needles, syringes, culture medium for one or more of the suitable hosts. Associated with the kits can be instructions customarily included in commercial packages of therapeutic or diagnostic products, that contain information about for example the indications, usage, dosage, manufacture, administration, contraindications and/or warnings concerning the use of such therapeutic or diagnostic products.

The invention encompasses the use of a binding molecule capable of binding to ALCAM, such as a binding molecule as described herein, in a process of diagnosing AML.

The present invention also provides the use of a binding molecule capable of binding to ALCAM for the preparation of a medicament for the prevention, treatment or combination thereof of AML. In other words, the invention also relates to a method of diagnosis, prevention and/or treatment of AML by administration of a binding molecule capable of binding to ALCAM. The human binding molecules of the invention are particularly useful, and often preferred, when to be administered to human beings as in vivo therapeutic agents, since recipient immune response to the administered antibody will often be substantially less than that occasioned by administration of a monoclonal murine, chimeric or humanised binding molecule. Routes of administration of AML by binding molecules or pharmaceutical compositions comprising such for prevention and/or therapy are well-known to the skilled artisan.

The binding molecules or pharmaceutical compositions of the invention may be employed in conjunction with other molecules useful in diagnosis, prevention and/or treatment of AML. They can be used in vitro, ex vivo or in vivo. The molecules are typically formulated in the compositions and pharmaceutical compositions of the invention in a therapeutically or diagnostically effective amount. Dosage regimens can be adjusted to provide the optimum desired response (e.g., a therapeutic response). For example, a single bolus may be administered, several divided doses may be administered over time or the dose may be proportionally reduced or increased as indicated by the exigencies of the therapeutic situation. The molecules and compositions according to the present invention are preferably sterile. Methods to render these molecules and compositions sterile are well known in the art. The other molecules useful in diagnosis, prevention and/or treatment can be administered in a similar dosage regimen as proposed for the binding molecules of the invention. If the other molecules are administered separately, they may be adminstered to a subject with AML prior to, concomitantly with, or subsequent to the administration of one or more of the binding molecules or pharmaceutical compositions of the invention. The dosing regimen is usually sorted out during clinical trials in human patients.

### EXAMPLES

To illustrate the invention, the following examples are provided. These examples are not intended to limit the scope of the invention.

### Example 1

### Selection of Phages Carrying Single Chain Fv Fragments Specifically Recognizing Human Acute Myeloid Leukemia Cells

Antibody fragments were selected using antibody phage display libraries, general phage display technology and MAbstract® technology, essentially as described in U.S. Pat. No. 6,265,150 and in WO 98/15833 (both of which are incorporated by reference herein). Furthermore, the methods and helper phages as described in WO 02/103012 (incorporated by reference herein) were used in the present invention. For identifying phage antibodies recognizing AML tumor cells phage selection experiments were performed using the acute myeloid leukemia cell line NB4 and primary AML tumor cells that were obtained from bone marrow aspirates of AML patients.

An aliquot of a phage library (500 μl, approximately 10¹³ cfu, amplified using CT helper phage (see WO 02/103012)) was blocked and presubtracted by mixing the library with 10 ml of RPMI 1640 medium with 10% FBS containing 230*10⁶ peripheral blood leukocytes (PBL). The obtained mixture was rotated at 4° C. for 1.5 hours. Hereafter, the cells were pelleted and the supernatant containing the phage library was transferred to a new tube containing a fresh pellet of 230*10⁶ PBL. The cells were resuspended in the phage library supernatant and the mixture was again rotated at 4° C. for 1.5 hours. This procedure was repeated once more and eventually 10 ml of supernatant containing the blocked phage library, which was 3 times subtracted with PBL, was transferred to a new tube and was kept overnight at 4° C. The next day 4*10⁶ cells of the acute promyelocytic leukaemia cell line called NB4 were pelleted in a separate 15 ml tube and the cells were resuspended in 1 ml of RPMI 1640 medium with 10% FBS. To the tube 3.3 ml of the presubtracted blocked phage library and 5 ml of RPMI 1640 medium with 10% FBS was added and the mixture was rotated at 4° C. for 2 hours. Hereafter, the obtained mixture was transferred to a 50 ml tube and washed 5 times with 30 ml RPMI 1640 medium with 10% FBS. To the pelleted cells 0.8 ml of 50 mM glycine-HCl pH 2.2 was added, mixed well and left at room temperature for 10 minutes to elute the attached phages. After that, 0.4 ml of 1 M Tris-HCl pH 7.4 was added for neutralization. Then, the cells were pelleted again and the supernatant was used to infect 5 ml of a XL1-Blue E. coli culture that had been grown at 37° C. to an OD600nm of approximately 0.3. The phages were allowed to infect the XL1-Blue bacteria for 30 minutes at 37° C. Subsequently, the mixture was centrifuged for 10 minutes, at 3200* g at room temperature and the bacterial pellet was resuspended in 1 ml 2-trypton yeast extract (2TY) medium. The obtained bacterial suspension was divided over a 2TY agar plate supplemented with tetracyclin, ampicillin and glucose. After incubation overnight of the plates at 37° C., the colonies were scraped from the plates and used to prepare an enriched phage library, essentially as described by De Kruif et al. (1995a) and WO 02/103012. Briefly, scraped bacteria were used to inoculate 2TY medium containing ampicillin, tetracycline and glucose and grown at a temperature of 37° C. to an OD600 nm of ˜0.3. CT helper phages were added and allowed to infect the bacteria after which the medium was changed to 2TY containing ampicillin, tetracycline and kanamycin. Incubation was continued overnight at 30° C. The next day, the bacteria were removed from the 2TY medium by centrifugation after which the phages in the medium were precipitated using polyethylene glycol (PEG) 6000/NaCl. Finally, the phages were dissolved in 2 ml of phosphate buffered saline (PBS) with 1% bovine serum albumin (BSA), filter-sterilized and used for the next round of selection. To this purpose a 500 μl aliquot of the NB4-derived amplified sublibrary was blocked with 2 ml of RPMI 1640 medium with 10% FBS for 30 minutes at 4° C. To the blocked sublibrary 5×10⁶ thawed primary AML blasts (90% CD33+ CD34+ blasts, FAB type M0) were added that previously had been stained with a PE-labelled anti-CD34 antibody (Becton Dickinson). The obtained mixture rotated at 4° C. for 2.5 hours. Hereafter, the mixture was transferred to a 50 ml tube, washed 3 times with 30 ml cold RPMI 1640 medium with 10% FBS. Subsequently, the mixture was passed over a 70 micron cell strainer and was subjected to flow cytometry. Cell sorting was performed using a FACSVantage flow cytometer (Becton Dickinson). Cells were gated on the basis of low sideward scatter (SSC) combined with CD34-PE staining. Approximately 9*10⁵ cells were sorted. The sorted cells were spun down, the supernatant was saved and the bound phages were eluted from the cells by resuspending the cells in 800 μl 50 mM glycin-HCl pH 2.2 followed by incubation for 5 minutes at room temperature. The obtained mixture was neutralized with 400 μl 1 M Tris-HCl pH 7.4 and added to the rescued supernatant. The eluted phages were used to re-infect XL1-Blue E. coli cells as described supra. After the second round of selection, individual E. coli colonies were used to prepare monoclonal phage antibodies. Essentially, individual colonies were grown to log-phase in 96 well plate format and infected with CT helper phages after which phage antibody production was allowed to proceed overnight. The produced phage antibodies were PEG/NaCl-precipitated and filter-sterilized and tested using flow cytometry (FACSCalibur, Becton Dickinson) for binding to both the NB4 acute promyelocytic leukaemia cell line as well as to the primary AML blasts (that were used for the second round selection). From the selection a large panel of phage antibodies was obtained that demonstrated binding to NB4 cells and AML blasts as well as to various subsets of peripheral blood leukocytes. One of the selected phage antibodies, called SC02-407, bound to both the primary AML tumor blasts as well as to NB4 acute promyelocytic leukemia cells and was analyzed in further detail.

### Example 2

### Characterization of scFv SC02-407

Plasmid DNA was obtained from the selected scFv clone SC02-407 according to standard techniques known in the art. Thereafter, the nucleotide sequence of scFv clone SC02-407 was determined according to standard techniques well known to a person skilled in the art. The nucleotide sequence of SC02-407 is listed in Table 1 and has SEQ ID NO:1. The amino acid translation of the nucleotide sequence is also listed in Table 1 and has SEQ ID NO:2. The VH and VL gene identity and amino acid sequence of the heavy chain CDR3 region of SC02-407 (see SEQ ID NO:3) are also depicted in Table 1.

### Example 3

### Expression of the Antigen Recognized by SC02-407 on Primary AML Samples, Tumor Cell Lines and Normal Hematopoetic Cells

The distribution of the target antigen recognized by the SC02-407 phage antibody was analyzed by flow cytometry using primary AML samples, tumor cell lines and normal hematopoetic cells derived peripheral blood.

For flow cytometry analysis, phage antibodies were first blocked in an equal volume of PBS, 4% milkprotein (MPBS) for 15 minutes at 4° C. prior to the staining of the various cells. The binding of the phage antibodies to the cells was visualized using a biotinylated anti-M13 antibody (Santa Cruz Biotechnology) followed by addition of streptavidin-allophycocyanin or streptavidin-phycoerythrin (Caltag).

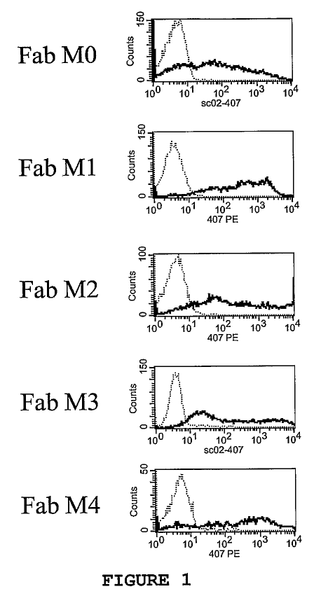

The CD45-positive blast population of a set of five different primary AML blasts (FAB subtypes: AML 11, FAB-M0; AML3, FAB-M1; AML98028, FAB-M2; AML97028, FAB-M3; AML98081, FAB-M4) was analyzed for binding of the SC02-407 phage antibody in a direct comparison with CD33 expression. Phage antibody SC02-407 showed strong binding to primary AML blasts of all subtypes tested as compared to a control phage antibody SC02-428 (see FIG. 1).

Analysis of a panel of tumor cell lines of both hematopoetic and non-hematopoetic origin revealed that expression of the antigen recognized by SC02-407 was not restricted to a subset of tumor cell lines of myeloid origin, i.e. HL-60 and NB4, since it also reacted with cell lines of epithelial origin (see Table 2).

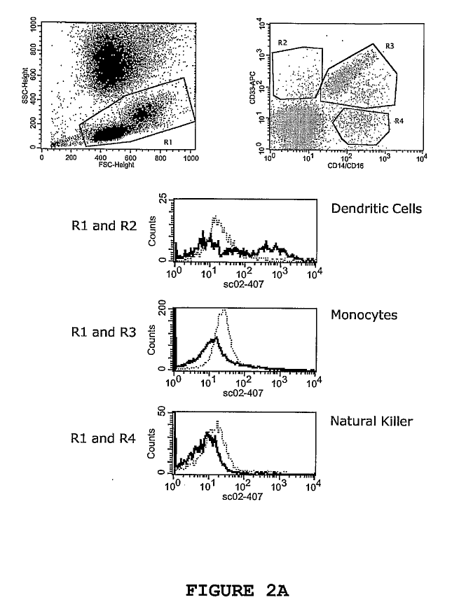

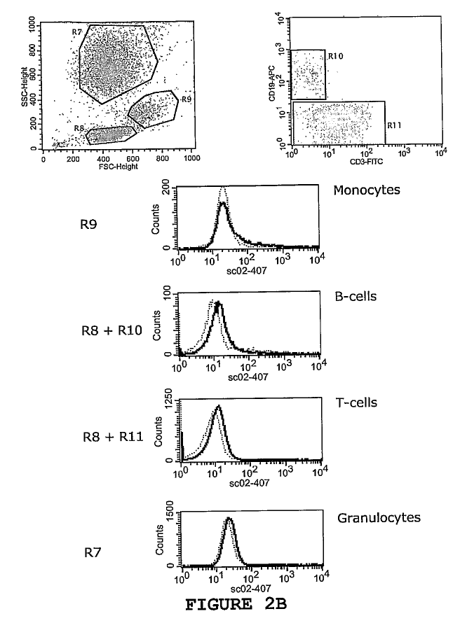

Within peripheral blood, subpopulations were analyzed by flow cytometry by staining with antibodies recognizing the cell surface antigens CD14 (FITC-labeled, Becton Dickinson), CD16 (FITC-labeled, Pharmingen) and CD33 (APC-labelled, Becton Dickinson) (see FIG. 2A). As shown in FIG. 2A, SC02-407 does recognize a subpopulation of dendritic cells, but does not recognize monocytes and natural killer (NK) cells.

Flow cytometric analysis was also performed by gating the lymphocyte-, monocyte- and granulocyte subpopulations on the basis of their forward- and side scatter characteristics. The lymphocytes were further divided in B-cells and T-cells by staining the sample with an APC-conjugated anti-CD19 antibody (Pharmingen) and a FITC-conjugated anti-CD3 antibody (Becton Dickinson). As shown in FIG. 2B SC02-407 does not recognize monocytes and almost no staining could be observed with B- and T-cells and granulocytes.

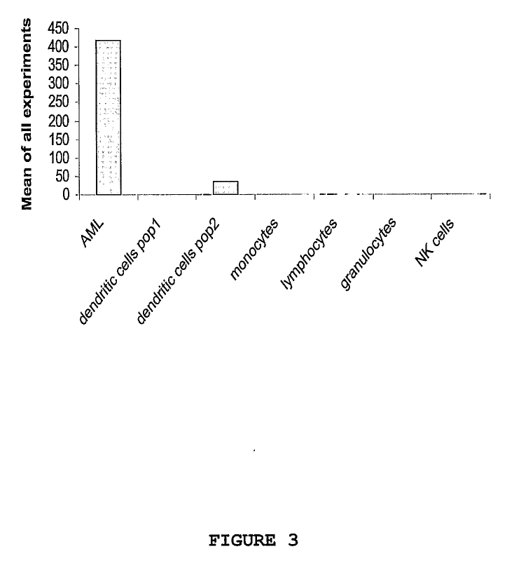

In FIG. 3 is shown that the binding intensity of the phage antibody SC02-407 to AML is much higher than the binding intensity of the phage antibody to different cell populations in peripheral blood of a healthy donor. The mean fluorescence of SC02-407 was calculated for AML and the different cell populations. Furthermore, the mean fluorescence of a control antibody (called SC02-006 and binding to thyroglobulin) was calculated for AML and the different cell populations (data not shown) and this value was deducted from the value of SC02-407.

From these combined expression data we concluded that SC02-407 recognizes a marker present on AML cells. The marker was to a minor amount also detectable on dendritic cells and on cell lines of myeloid origin and epithelial origin.

### Example 4

### Generation of 407 IgG1 Molecules

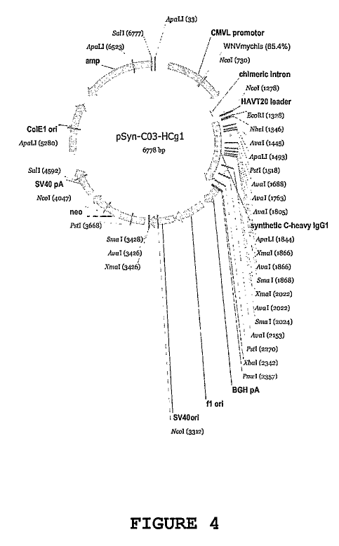

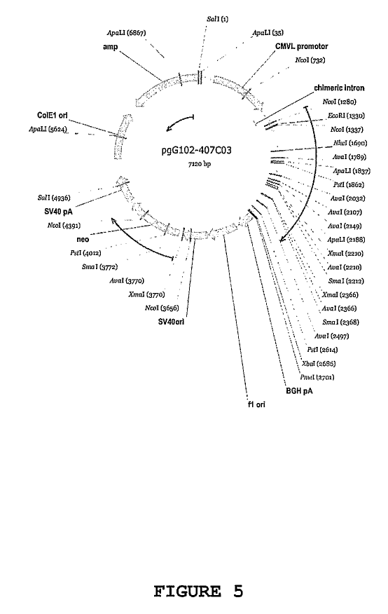

Heavy- and light chain variable regions of the scFv's SC02-407 were PCR-amplified using oligonucleotides to append restriction sites and/or sequences for expression in the IgG expression vectors pSyn-C03-HCgl (see FIG. 4; SEQ ID NO:4) and pcDNA3.1 (containing a CMV long promoter).

The VL chain was amplified using the oligonucleotides 5L-A (SEQ ID NO:6) and 3L-B (SEQ ID NO:7). The PCR products were cloned into vector pcDNA3.1 containing a CMV long promoter and the nucleotide sequences were verified according to standard techniques known to the skilled artisan. The resulting expression construct was called pSyn-C04-Vlambda3.

VH genes were amplified using oligonucleotides 5H-B (SEQ ID NO:8) and Sy3H-a reversed (SEQ ID NO:9). Thereafter, the PCR products were cloned into vector pSyn-C03-HCgl and nucleotide sequences were verified according to standard techniques known to the skilled person in the art. The resulting expression construct was called pgG102-407C03 (See FIG. 5).

5H-B
acctgtcttgaattctccatggcccaggtgcagctgcaggagtccggccc
Sy3H-a reversed
ggggccagggcaccctggtgaccgtctccagcgctagcaccaagggc
5L-A
acctgtctcgagttttccatggcttcctccgagctgactcaggaccctgc
tg
3L-B
gaccaagctgaccgtcctaggtgagtcgcggccgctaaggaaaa

The resulting expression construct pgG102-407C03 was transiently expressed in combination with the pSyn-C04-Vlambda3 construct (see FIG. 6 and SEQ ID NO:5) encoding the light chain in 293T cells and supernatants containing IgG1 antibodies were obtained. The nucleotide sequence of the heavy chain of the antibody called CR2407 is shown in SEQ ID NO:10. The amino acid sequences of the heavy chain of the antibody called CR2407 is shown in SEQ ID NO:11. The nucleotide sequence of the light chain of the antibody called CR2407 is shown in SEQ ID NO:12. The amino acid sequences of the light chain of the antibody called CR2407 is shown in SEQ ID NO:13.

The antibodies were purified on protein-A columns and size-exclusion columns using standard purification methods used generally for immunoglobulins (see for instance WO 00/63403).

### Example 5

### Immunoprecipitation of a Membrane Extractable Antigen Recognized by CR2407.

To identify whether CR2407 reacted with a membrane extractable antigen, the cell surface of 10⁸ HEp-2 cells were biotinylated during 1 hour at room temperature with a final concentration of 2 mg sulfo-NHS-LC-LC-biotin in physiological buffer (0.2 M phosphate buffer containing 0.12 M NaCl, pH 7.4). Subsequently, the remaining free biotin was blocked during an incubation of 30 minutes at room temperature with 10 mM glycine in physiological buffer. After labeling, the cells were washed with cold physiological buffer and solubilized for 30 minutes on ice at a concentration of 3×10⁷ cells/ml in TX-100 lysis buffer (1% Triton X-100, 150 mM NaCl, 50 mM Tris pH 7.4, protease inhibitors (Roche)). The unsoluble material was removed by centrifugation for 30 minutes at 4° C. at 20,000*g.

Hereafter, the biotinylated solubilized lysates were pre-cleared with protein-A beads for 2 hours at 4° C. 4 μg of CR2407 and the negative control antibody CR2428 and positive control antibody CR2300 (directed against CD46) were coupled to protein A beads at room temperature. Next, the pre-cleared samples were incubated with the above IgG's coupled to protein-A beads for 2 hours at 4° C. The protein-A beads were washed three times for 5 minutes with 1 ml of TX-100 lysis buffer and bound complexes were eluted by the addition of sample loading buffer. The samples were subjected to SDS-PAGE under non-reducing and reducing conditions. After blotting on PVDF membranes, the biotinylated proteins were detected with streptavidin-HRP (Amersham) and enhanced chemoluminescence (Amersham).

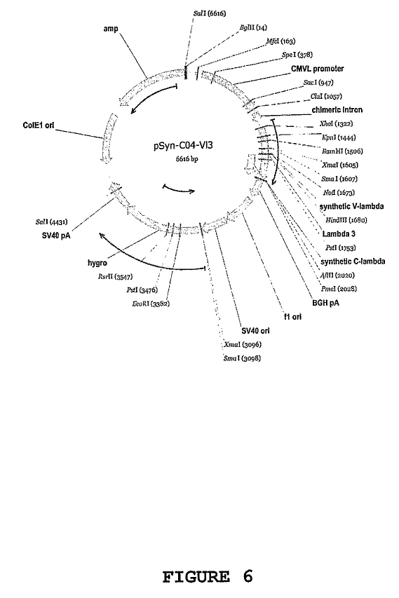

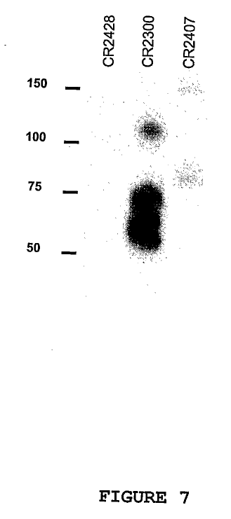

After immunoprecipitation of HEp-2 cell lysate with CR2407 and non-reducing SDS-PACE one broad band of 80-90 kDa and one sharp band of 150 kDa could be observed (see FIG. 7). These bands were not present in immunoprecipitations performed with negative control IgG1 (CR2428) or human IgG1 directed against CD46 (CR2300).

To establish optimal wash and elution conditions for the big scale purification imnunecomplexes of CR2407 immunoprecipitates were subjected to different washing conditions with different extraction buffers including concentrations of NaCl varying from 150 mM-500 mM in the above mentioned TX-100 lysis buffer. Furthermore, immunecomplexes were eluted off the protein A beads using low glycine (pH 2.6) or high lysine (pH 11) buffers. The immunecomplexes did not elute in high salt environment (500 mM NaCl in RIPA buffer (1% Triton X-100, 0.5% desoxycholate, 0.1% SDS, 50 mM Tris pH 7.4, protease inhibitors (Roche))), whereas they became eluted with a low pH buffer (pH 2.6) (data not shown).

### Example 6

### Purification and Identification of the Antigen Reacting with CR2407

For the purification of the antigen of CR2407 an affinity column was prepared by coupling 1.5 mg CR2407 to 1 ml CNBr activated Sepharose-4B beads according to standard techniques known to the skilled artisan. In advance, the IgG1 antibody was passed over a 100 kDa ultracentrifugal device to remove incomplete, small IgG fragments.

A cell lysate of 5*10⁹ HEp-2 cells was prepared in TX-100 buffer buffer according to the method described in example 5. Next, the cell lysate was passed through a 0.22 μM filter to remove aggregates. The cell lysate was pre-cleared for 4 hours at 4° C. with 60 ml blocked CNBr-activated sepharose CL-4B beads, followed by pre-clearing for 4 hours at 4° C. with 5 ml of CNBr-activated beads to which human control IgG1 was coupled (1 mg IgG1/ml, Cappel) to clear the lysates from proteins that interact aspecifically with IgG. Next, the individual lysates were passed through a 0.22 μM filter to remove insoluble material. The affinity columns of the negative control antibody CR2428 and the antibody CR2407 were connected in series (column comprising control antibody first) to an AKTA FPLC-900 and equilibrated with TX-100 buffer. The lysates were applied to the columns at 1 ml/min and columns were washed individually with 5 column volumes TX-100, 5 column washes with RIPA buffer, followed by a salt gradient in TX-100 buffer from 150 mM NaCl to 500 mM NaCl, a wash with 2 column volumes TX-100 buffer and an elution of 5 column volumes glycine pH 2.6, whereby after 1 column volume of elution buffer, the flow through was put for 10 minutes on hold to enhance the release of the immunecomplexes. Next, the column was washed with 5 column volumes of TX-100 buffer. The eluted fractions of 0.5 ml were neutralized with 20 μl 2 M Tris/HCl of pH 7.4, and 20 μl of the samples were run on a non-reducing SDS-PAGE criterion gel and stained with Silver Stain according to standard techniques known to the skilled artisan. The SDS-PAGE profile of the proteins eluting from the CR2407 column showed that the expected protein with a molecular weight of 80-90 kDa became specifically released from the column in fraction 28 (data not shown). Fraction 28 contained in addition two other protein bands, one of 50 kDa and a protein of 60-70 kDa (data not shown). Next, fraction 28 was 5 times concentrated using YM-filters and loaded on a non-reducing SDS-PAGE gel. The 80-90 kDa band (which corresponds with one of the bands found after immunoprecipitation of HEp-2 cell lysate with CR2407) was cut out from the gel with a sharp razor and subjected to mass spectrometry analysis by nano-electrospray ionization tandem MS (nanoESI-MS-MS) performed at MDS (Odense, Denmark). The analysis was performed essentially as described in Shevchenko et al. (1996). Using nanoESI-MS-MS a peptide having an amino acid sequence of SSNTYTLTDVR (SEQ ID NO:14) was identified. This peptide was identified by blast analysis as being part of the human protein MEMD (see accession number CAA71256; gi3183975 in the NIH BLAST database) for the human MEMD protein, which is identical to the human protein ALCAM (for amino acid sequence of human MEMD/ALCAM see SEQ ID NO:15).

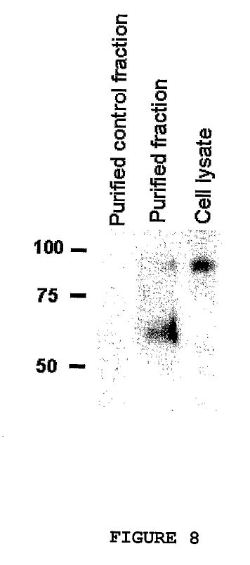

To confirm the identification of ALCAM as the antigen recognized by CR2407 the purified fraction (fraction 28), a purified negative control fraction (fraction 22) and a positive cell lysate (Hep-2 cell lysate) were analyzed for the presence of ALCAM using a commercially available murine monoclonal antibody specific for ALCAM. The samples were subjected to SDS-PAGE under non-reducing conditions. After blotting on PVDF membranes, the membranes were placed in TBST containing 4% non-fat milk powder (TBST/milk) and incubated with a murine monoclonal antibody directed against ALCAM (Monosan) (1 μg/ml in TBST/milk) for 1 hour at room temperature followed by washing 3 times for 5 minutes in TBST. Next, the membranes were incubated with horseradish conjugated rabbit anti-mouse antibody (DAKO) 1 μg/ml (in TBST/milk) for one hour at room temperature. Finally, the membranes were washed extensively in TBST followed by a PBS washing step. Reactive proteins were revealed by a chemofluorescence detection system (ECL). As demonstrated in FIG. 8, ALCAM was detected in the purified fraction and the cell lysate and was absent in the control fraction. In addition to the protein with the expected molecular weight of 80-90 kDa, a 60 kDa protein was detected in the purified fraction. This protein might be a proteolytic cleavage product of mature ALCAM.

### Example 7

### Expression of ALCAM in AML

The expression of ALCAM in a large set of AML samples was analyzed by flow cytometry using the phage antibody SC02-407. To simplify the FACS analysis phage antibodies instead of IgGs were used, since phage antibodies in contrast to IgG molecules do not bind to the Fc receptors present on the AML blasts. Phage antibody staining was performed as described supra. The following antibody combinations were used in the analysis: CD45-PerCP, indirect labeling of SC02-407 with anti-M13 biotin and streptavidin-PE (as described in Example 3 supra), and CD33-allophycocyanin (APC). The cells were washed twice with PBS containing 1% BSA and resuspended in binding buffer and dead/apoptotic cells were excluded by a staining with Annexin V-FITC in annexin V binding buffer. Cells were analysed on a FACS calibur (BD) using CellQuest software. For final analysis blasts cells were gated based on low side scatter versus CD45 expression. A sample was considered positive if more than 20% of the blast cells (CD45+ cells) expressed ALCAM (compared to the control sample).

As summarized in Table 3, ALCAM was expressed on more than 20% of blast cells in 89% of the 27 AML samples investigated and was detectable in AML samples throughout the different FAB subtypes analyzed. The analyzed samples included two CD33-negative AML samples. Both samples expressed ALCAM. The AML samples negative for ALCAM were all reactive with the anti-CD33 antibody. Therefore, ALCAM and CD33 might complement each other in the diagnosis of AML.

TABLE 1
Nucleotide and amino acid sequence of SC02-407 and
heavy chain CDR3 and the VH and VL gene identity.

| | SEQ ID NO | SEQ ID NO | | | |
|---|---|---|---|---|---|
| | of nucl. | of amino | | VH- | VL- |
| scFv | Seq. | acid seq | CDR3 | germline | germline |
| SC02-407 | SEQIDNO: 1 | SEQIDNO: 2 | GSLLQF | 3-23 | V13 |
| | | | (SEQIDNO: 3) | (DP-47) | (31-V2-13) |

TABLE 2
Analysis of tumor cell lines of hematopoetic and non-hematopoetic
origin for reactivity with SC02-407.

| Cell | | |
|---|---|---|
| line | Origin | SC02-407 reactivity |
| HL-60 | Acute Myeloid Leukemia | +/− |
| NB4 | Acute Promyelocytic Leukemia | ++ |
| U937 | Histiocytic Lymphoma | − |
| K562 | Erythroid Leukemia | − |
| 293T | Embryonal Kidney | ++ |
| LS174T | Colon Adenocarcinoma | ++ |
| HEp-2 | Cervix Epithelial cells | + |

reactivity − = <5%; reactivity 5-25% = +/−; reactivity 25-75% = +; reactivity <75% = ++

TABLE 3
Flow cytometry analysis of expression
of ALCAM in AML samples.

| | | |
|---|---|---|
| AML cells | | |
| FAB classification | ALCAM % positives | CD33% positives |

| M0 | 100 | (1^{#}/1*) | 100 | (1/1) |
|---|---|---|---|---|
| M1 | 75 | (3/4) | 100 | (4/4) |
| M1/2 | ND | | ND | |
| M2 | 100 | (4/4) | 100 | (4/4) |
| M3 | 100 | (1/1) | 100 | (1/1) |
| M4 | 80 | (4/5) | 100 | (5/5) |
| M5 | 100 | (4/4) | 75 | (3/4) |
| M5a | 100 | (3/3) | 100 | (3/3) |
| M5b | 100 | (1/1) | 100 | (1/1) |
| unclassified | 75 | (3/4) | 75 | (3/4) |
| total | 89 | (24/27) | 93 | (25/27) |

^{#}number of positive cases; a sample was considered positive if more than 20% of the blast population stained with SC02-407 or anti-CD33 antibody.
*number of cases tested
ND: not determined

### REFERENCES

Boel E, Verlaan S, Poppelier M J, Westerdaal N A, Van Strijp J A and Logtenberg T (2000), Functional human monoclonal antibodies of all isotypes constructed from phage display library-derived single-chain Fv antibody fragments. J. Immunol. Methods 239:153-166.

Burton D R and Barbas C F (1994), Human antibodies from combinatorial libraries. Adv. Immunol. 57:191-280.

De Kruif J, Terstappen L, Boel E and Logtenberg T (1995a), Rapid selection of cell subpopulation-specific human monoclonal antibodies from a synthetic phage antibody library. Proc. Natl. Acad. Sci. USA 92:3938.

De Kruif J, Boel E and Logtenberg T (1995b), Selection and application of human single chain Fv antibody fragments from a semi-synthetic phage antibody display library with designed CDR3 regions. J. Mol. Biol. 248:97.

Huls G, Heijnen I J, Cuomo E, van der Linden J, Boel E, van de Winkel J and Logtenberg T (1999), Antitumor immune effector mechanisms recruited by phage display-derived fully human IgG1 and IgA1 monoclonal antibodies. Cancer Res. 59: 5778-5784.

Jennings C D and Foon K A (1997), Recent advances in flow cytometry: application to the diagnosis of hematologic malignancy. Blood 90: 2863-2892.

Ohneda O, Ohneda K, Arai F, Lee J, Miyamoto T, Fukushima Y, Dowbenko D, Lasky L A and Suda T (2001), ALCAM (CD166): its role in hematopoietic and endothelial development. Blood 98: 2134-2142.

Shevchenko A, Jensen O N, Podtelejnikov A V, Sagliocco F, Wilm M, Vorm O, Mortensen P, Shevchenko A, Boucherie H and Mann M (1996), Linking genome and proteome by mass spectrometry: large-scale identification of yeast proteins from two dimensional gels. Proc. Natl. Acad.Sci. U S A. Dec 93:14440-14445.

van Kempen L C, van den Oord J J, van Muijen G N, Weidle U H, Bloemers H P and Swart G W (2000), Activated leukocyte cell adhesion molecule/CD166, a marker of tumor progression in primary malignant melanoma of the skin. Am. J. Pathol. 156:769-74.

1. A method of investigating the likelihood that an individual suffers from acute myeloid leukemia (AML), the method comprising the steps of:
a) contacting a sample comprising peripheral blood cells or bone marrow cells of the individual with a diagnostically effective amount of a binding molecule capable of binding to activated leukocyte cell adhesion molecule (ALCAM) and a diagnostically effective amount of a binding molecule capable of binding to CD33, and
b) determining whether at least one of the binding molecules specifically binds to cells in the sample.

2. A method according to claim 1, characterized in that the binding molecule capable of binding to ALCAM is contacted with the sample before, during, or after the step of contacting of the sample with the binding molecule capable of binding to CD33.

3. A method according to claim 1, characterized in that the AML is selected from the group consisting of the subtypes FAB-M0, FAB-M1, FAB-M2, FAB-M3, FAB-M4, FAB-M5, and unclassified AML.

4. The method according to claim 1, wherein the binding molecule capable of binding to ALCAM is a human monoclonal antibody.

5. A method according to claim 4, characterized in that the human binding molecule comprises at least a CDR3 region comprising the amino acid sequence of SEQ ID NO:3.

6. The method according to claim 1, wherein the binding molecules further comprise at least one tag.

7. A method according to claim 4, characterized in that the binding molecules comprise a different tag.

8. The method according to claim 6, characterized in that the tag is a fluorescent molecule.

9. The method according to claim 1, wherein the method is performed by flow cytometry.

10. A method according to claim 9, characterized in that the method is performed by using a fluorescence activated cell sorter.

11. The method according to wherein the method further comprises
a) identifying malignant cells in the sample before, during or after the step of contacting a sample comprising peripheral blood cells or bone marrow cells of an individual with a diagnostically effective amount of at least one of the binding molecules, and
b) determining whether at least one of the binding molecules specifically binds to a malignant cell.

12. A method according to claim 11, characterized in that the malignant cells are identified by flow cytometry.

13. method according to claim 1, wherein the individual is selected from the group consisting of an individual suspected of having AML, an individual having AML, an individual recovered from AML, and an individual relapsed from AML.

14. A human binding molecule capable of specifically binding to ALCAM, characterized in that the human binding molecule comprises at least a CDR3 region comprising the amino acid sequence of SEQ ID NO:3.

15. A human binding molecule according to claim 14, characterized in that the binding molecule comprises a heavy chain comprising the amino acid sequence of SEQ ID NO:11.

16. An immunoconjugate comprising the human binding molecule of claim 14 and a tag.

17. A nucleic acid molecule encoding the human binding molecule of claim 14.

18. A vector comprising at least one nucleic acid molecule according to claim 17.

19. A host cell comprising at least one vector according to claim 18.

20. A host cell according to claim 19, characterized in that the host cell is derived from a human cell.

21. A process of diagnosing acute myeloid leukemia (AML) in a subject, said process comprising:
obtaining a sample from the subject, and
interacting the sample with a binding molecule capable of binding to ALCAM together with a binding molecule capable of binding to CD33 so as to diagnose AML.

22. A pharmaceutical composition comprising: a human binding molecule capable of binding to ALCAM together with a binding molecule capable of binding to CD33.
